(19) United States
(12) Patent Application Publication
(10) Pub. No.: US 2024/0415869 A1
(43) Pub. Date: Dec. 19, 2024

(54) COMPOSITIONS, SYSTEMS, AND METHODS FOR TREATING CANCER USING TUMOR TREATING FIELDS (TTFIELDS) AND THIOREDOXIN/GLUTATHIONE INHIBITORS

(71) Applicant: Novocure GmbH G, Root D4 (CH)

(72) Inventors: Rotem Engelman, Haifa (IL); Bassam Abdulkarim, Montreal (CA)

(73) Assignee: Novocure GmbH, Root D4 (CH)

(21) Appl. No.: 18/743,392

(22) Filed: Jun. 14, 2024

Related U.S. Application Data
(60) Provisional application No. 63/508,346, filed on Jun. 15, 2023.

(51) Int. Cl. A61K 31/7135; A61N 1/36; A61P 35/00

(52) U.S. Cl. CPC A61K 31/7135; A61N 1/36002; A61P 35/00

(57) ABSTRACT

Compositions, kits, systems, and methods for reducing viability of cancer cells and treating cancer, as well as reducing a volume of a tumor and/or preventing an increase in volume of a tumor present in a body of a living subject, are disclosed. Also disclosed are methods of increasing reactive oxygen species (ROS) in cancer cells, along with methods of enhancing cytotoxicity of at least one thioredoxin reductase inhibitor and/or at least one glutathione inhibitor against cancer cells. The systems and methods involve application of an alternating field in combination with administration of at least one composition comprising at least one thioredoxin reductase inhibitor and/or at least one glutathione inhibitor.

### CROSS-REFERENCE TO RELATED APPLICATIONS

This application claims benefit under 35 USC 119 (e) of U.S. Provisional Application No. 63/508,346, filed Jun. 15, 2023. The entire contents of the above-referenced patent application(s) are hereby expressly incorporated herein by reference.

### BACKGROUND

Tumor Treating Fields (TTFields) are low intensity (e.g., 1-3 V/cm) alternating electric fields within the intermediate frequency range (such as, but not limited to, 100-500 kHz) that target solid tumors by disrupting mitosis. This non-invasive treatment targets solid tumors and is described, for example, in U.S. Pat. Nos. 7,016,725; 7,089,054; 7,333,852; 7,565,205; 8,244,345; 8,715,203; 8,764,675; 10,188,851; and 10,441,776. TTFields are typically delivered through two pairs of transducer arrays that generate perpendicular fields within the treated tumor; the electrode arrays that make up each of these pairs are positioned on opposite sides of the body part that is being treated. More specifically, for the OPTUNE® system, one pair of electrodes is located to the left and right (LR) of the tumor, and the other pair of electrodes is located anterior and posterior (AP) to the tumor. TTFields are approved for the treatment of glioblastoma multiforme (GBM), and may be delivered, for example, via the OPTUNE® system (Novocure Limited, St. Helier, Jersey), which includes transducer arrays placed on the patient's shaved head.

Each transducer array used for the delivery of TTFields in the OPTUNE® device comprises a set of ceramic disk electrodes, which are coupled to the patient's skin (such as, but not limited to, the patient's shaved head for treatment of GBM) through a layer of conductive medical gel. The purpose of the medical gel is to deform to match the body's contours and to provide good electrical contact between the arrays and the skin; as such, the gel interface bridges the skin and reduces interference. The device is intended to be continuously worn by the patient for 2-4 days before removal for hygienic care and re-shaving (if necessary), followed by reapplication with a new set of arrays. As such, the medical gel remains in substantially continuous contact with an area of the patient's skin for a period of 2-4 days at a time. In addition, the arrays can be shifted a few centimeters in either direction to allow the skin to heal from one period of treatment to the next. Therefore, a portion of skin that was covered by electrodes/gel for a 2-4 day period could then be uncovered for 2-4 days when the replaced electrodes are shifted slightly; then the device may be reapplied to the original portion of skin for the next 2-4 day period.

Recently, application of TTFields has been shown to induce reactive oxygen species (ROS) production (Rominiyi et al. (2021) Br J Cancer, 124 (4): 697-709; Jo et al. (2018) Int J Mol Sci., 19 (11): 3684; Liu et al. (2021) Biomedicine & Pharmacotherapy, 141:111810; Kim et al. (2016) Oncotarget, 7 (38): 62267-62279); Park et al. (2019) Technol Cancer Res Treat, 18:1-9).
Reactive Oxygen Species (ROS) are normal byproducts of numerous cellular processes, and there is a balance between ROS production and antioxidant defense. In order to maintain ROS levels in cells and prevent oxidative stress, there are several ROS scavengers, the main two being the thioredoxin (Trx) and glutathione (GSH) systems. ROS have a dual role in cell metabolism: at low to moderate levels, ROS act as signal transducers to activate cell proliferation, migration, invasion, angiogenesis, and drug resistance (i.e., aspects important for tumor development and progression); however, high levels of ROS can cause damage to proteins, DNA, and other organelles, leading to cell death. Typically, cancer cells exhibit higher basal levels of ROS compared with normal cells; therefore, in order to maintain cellular processes and cellular proliferation, cancer cells have elevated antioxidant defenses, largely through upregulation of the Trx and GSH systems.

The thioredoxin (Trx) system plays an important role in the regulation of intracellular redox balance, and overexpression of thioredoxin reductase 1 (TrxR1) inhibits ROS increase and correlates with chemo/radioresistance and aggressive disease in GBM. TrxR1 is up-regulated in more than half of GBMs, and TrxR1 overexpression in GBM counteracts RT (radiation therapy)-induced ROS, leading to radioresistance.

Auranofin (RIDAURA®, Prometheus Laboratories Inc., San Diego, CA) is a TrxR inhibitor that interacts with TrxR and inhibits its activity, thereby leading to ROS increase. Auranofin is FDA-approved for the treatment of Rheumatoid Arthritis. Auranofin is orally available, well-tolerated, and lipophilic, and Auranofin also crosses the blood-brain barrier.

The potential anticancer activity of auranofin has been recognized (see, for example, Onodera et al. (2019) Chem Pharm Bull (Tokyo), 67 (3): 186-191; and Szeliga et al. (2022) Int J Mol Sci, 23 (24): 15712), and the efficacy of auranofin has been tested against breast, lung, and leukemia cancer cell lines, and also tested in GBM (using the CUSP-9 treatment protocol). Preliminary results from these studies have shown that: (1) Auranofin (Au) decreased neurosphere formation ability in glioblastoma stem cells (GSCs) and clonogenic potential in GBM cell lines; (2) Auranofin decreased cell survival with lower IC₅₀s for GSCs compared to GBM cell lines; (3) Auranofin significantly increases intracellular ROS in GBM cell lines and GSCs; and (4) the combination of Auranofin with radiation therapy (RT, also known as radiotherapy) synergistically increases ROS in GSCs.

Together with the thioredoxin and Nicotinamide adenine dinucleotide phosphate (NADP/NADPH) systems, the glutathione/glutathione disulfide (GSH/GSSG) redox couple determines the redox state in biological systems and maintains intracellular redox potential. The deregulation of glutathione metabolism is broadly identifiable in the majority of cancers, as the genes involved in GSH turnover or utilization are under the transcriptional control of classical tumorigenic pathways. High GSH levels observed in many cancer cells allow the cells to cope with the oxidative stress caused by their increased metabolism and proliferation rate and protect them from the activity of chemotherapeutic agents. Therefore, inhibitors of GSH metabolism, such as (but not limited to) erastin, buthionine sulfoximine (BSO), sulfasalazine, BPTES B-839, and BSO sulfasalazine, have been tested as anti-cancer agents. While targeting glutathione metabolism has been widely investigated for cancer treatment, the depletion of glutathione using GSH inhibitors alone as a single therapeutic strategy has so far not proven very effective when compared to other, combinatorial approaches (Desideri et al. (2019) Nutrients 11 (8): 1926).

### BRIEF DESCRIPTION OF THE DRAWINGS

FIG. 1 illustrates an analysis of the effects of TTFields treatment on the expression of various genes in the thioredoxin and glutathione pathways in several cancer cell lines.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions, assemblies, systems, kits, and/or methods disclosed herein can be made and executed without undue experimentation in light of the present disclosure. While the compositions, assemblies, systems, kits, and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (e.g., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio.

The term “patient” or “subject” as used herein includes human and veterinary subjects. “Mammal” for purposes of treatment refers to any animal classified as a mammal, including (but not limited to) humans, domestic and farm animals, nonhuman primates, and any other animal that has mammary tissue.

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent/element/method to a patient for therapeutic and/or prophylactic/preventative purposes.

The term “therapeutic composition” or “pharmaceutical composition” as used herein refers to an agent that may be administered in vivo to bring about a therapeutic and/or prophylactic/preventative effect.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner, and can vary depending on factors known in the art, such as (but not limited to) the type of condition/disease/infection, the patient's history and age, the stage of the condition/disease/infection, and the co-administration of other agents.

The term “effective amount” refers to an amount of a biologically active molecule or conjugate or derivative thereof, or an amount of a treatment protocol (e.g., an alternating electric field), sufficient to exhibit a detectable therapeutic effect without undue adverse side effects (such as (but not limited to) toxicity, irritation, and allergic response) commensurate with a reasonable benefit/risk ratio when used in the manner of the inventive concept(s). The therapeutic effect may include, for example but not by way of limitation, preventing, inhibiting, or reducing the occurrence of at least one condition, disease, and/or infection. The effective amount for a subject will depend upon the type of subject, the subject's size and health, the nature and severity of the condition/disease/infection to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “concomitant therapy” and “adjunct therapy,” and will be understood to mean that the patient in need of treatment is treated or given another drug for the condition/disease/infection in conjunction with the treatments of the present disclosure. This concurrent therapy can be sequential therapy, where the patient is treated first with one treatment protocol/pharmaceutical composition and then the other treatment protocol/pharmaceutical composition, or the two treatment protocols/pharmaceutical compositions are given simultaneously. In addition, it will be understood that one administration step (such as, but not limited to, administration of the TTFields) may occur over a longer period of time than the other administration step (i.e., oral administration or injection of a substance). In these instances of varying administration time periods, the term “simultaneously” will be understood to mean that the shorter administration step wholly overlaps with the longer administration step. However, the term “simultaneously” will include performing the shorter administration step at any point during the longer administration step (e.g., the beginning, middle, or end of the longer administration step, or any other time period therebetween), as well as performing the shorter administration step one or more times wholly within the time period of the longer administration step. Therefore, the term “simultaneously” does not require that the two administration steps be performed over the exact same length of time.

The terms “administration” and “administering,” as used herein, will be understood to include all routes of administration known in the art, including but not limited to, oral, topical, transdermal, parenteral, subcutaneous, intranasal, mucosal, intramuscular, intraperitoneal, intravitreal, and intravenous routes, and including both local and systemic applications. In addition, the compositions of the present disclosure (and/or the methods of administration of same) may be designed to provide delayed, controlled, or sustained release using formulation techniques which are well known in the art.

The term “target region,” as used herein, refers to a region containing all or a portion of the cancer, cancer cells, and/or tumor to be treated.

Turning now to the inventive concept(s), a concurrent therapy for cancer is disclosed herein. The concurrent therapy includes the use of alternating electric fields (e.g., TTFields) in combination with at least one thioredoxin reductase inhibitor and/or at least one glutathione inhibitor. The combination of alternating electric fields (e.g., TTFields) with the at least one thioredoxin reductase inhibitor and/or at least one glutathione inhibitor provides a synergistic result in the treatment of cancer.

Certain non-limiting embodiments of the present disclosure are directed to a method of reducing viability of cancer cells. The method includes the steps of: (1) administering at least one composition to the cancer cells, wherein the at least one composition comprises at least one thioredoxin reductase inhibitor and/or at least one glutathione inhibitor; and (2) applying an alternating electric field to the cancer cells for a period of time.

Certain non-limiting embodiments of the present disclosure are directed to a method of enhancing the cytotoxicity of at least one thioredoxin reductase inhibitor and/or at least one glutathione inhibitor against cancer cells. The method includes the steps of: (1) administering at least one composition to the cancer cells, wherein the at least one composition comprises at least one thioredoxin reductase inhibitor and/or at least one glutathione inhibitor; and (2) applying an alternating electric field to the cancer cells for a period of time.

Certain non-limiting embodiments of the present disclosure are directed to a method of increasing reactive oxygen species (ROS) in cancer cells. The method includes the steps of: (1) administering at least one composition to the cancer cells, wherein the at least one composition comprises at least one thioredoxin reductase inhibitor and/or at least one glutathione inhibitor; and (2) applying an alternating electric field to the cancer cells for a period of time.

Certain additional non-limiting embodiments of the present disclosure are directed to a method of treating cancer in a subject. The method includes the steps of: (1) administering at least one composition to the subject, wherein the at least one composition comprises at least one thioredoxin reductase inhibitor and/or at least one glutathione inhibitor; and (2) applying an alternating electric field to a target region of the subject.

Certain additional non-limiting embodiments of the present disclosure are directed to a method of reducing a volume of a tumor, wherein the tumor is present in a body of a living subject and includes a plurality of cancer cells. The method includes the steps of: (1) administering at least one composition to the subject, wherein the at least one composition comprises at least one thioredoxin reductase inhibitor and/or at least one glutathione inhibitor; and (2) applying an alternating electric field to a target region of the subject.

Certain additional non-limiting embodiments of the present disclosure are directed to a method of preventing an increase of volume of a tumor, wherein the tumor is present in a body of a living subject and includes a plurality of cancer cells. The method includes the steps of: (1) administering at least one composition to the subject, wherein the at least one composition comprises at least one thioredoxin reductase inhibitor and/or at least one glutathione inhibitor; and (2) applying an alternating electric field to a target region of the subject.

Steps (1) and (2) of any of the methods of the present disclosure may be performed concomitantly or sequentially, and in particular, substantially simultaneously or wholly or partially sequentially. When the steps are performed wholly or partially sequentially, the at least one composition comprising at least one thioredoxin reductase inhibitor and/or at least one glutathione inhibitor may be administered before or after application of the alternating electric field has begun.

The methods of the present disclosure may be utilized to treat any types of cancer cells/cancers/tumors that respond to treatment with alternating electric fields (e.g., TTFields) and/or thioredoxin reductase/glutathione inhibitor(s). Non-limiting examples of cancer cells/cancers/tumors that can be treated in accordance with the present disclosure include hepatocellular carcinoma/carcinoma cells, glioblastoma/glioblastoma cells, pleural mesothelioma/mesothelioma cells, differentiated thyroid cancer/cancer cells, advanced renal cell carcinoma/carcinoma cells, ovarian cancer/cancer cells, breast cancer/cancer cells, pancreatic cancer/cancer cells, lung cancer/cancer cells (such as, but not limited to, non-small cell lung cancers), and the like, as well as any combination thereof.

In a particular (but non-limiting) embodiment, the cancer may be a solid tumor.

Any type of conductive or non-conductive electrode(s) and/or transducer array(s) that can be utilized for generating an alternating electric field that are known in the art or otherwise contemplated herein may be utilized for generation of the alternating electric field in accordance with the methods of the present disclosure. Non-limiting examples of electrodes and transducer arrays that can be utilized for generating an alternating electric field in accordance with the present disclosure include those that function as part of an alternating electric field-generating system (e.g., TTFields system) as described, for example but not by way of limitation, in U.S. Pat. Nos. 7,016,725; 7,089,054; 7,333,852; 7,565,205; 8,244,345; 8,715,203; 8,764,675; 10,188,851; and 10,441,776; and in US Patent Application Publication Nos. US 2018/0160933; US 2019/0117956; US 2019/0307781; and US 2019/0308016.

The alternating electric field may be generated at any frequency in accordance with the present disclosure. For example (but not by way of limitation), the alternating electric field may have a frequency of about 50 kHz, about 60 kHz, about 70 kHz, about 75 kHz, about 80 kHz, about 90 kHz, about 100 kHz, about 105 kHz, about 110 kHz, about 115 kHz, about 120 kHz, about 125 kHz, about 130 kHz, about 135 kHz, about 140 kHz, about 145 kHz, about 150 kHz, about 155 kHz, about 160 kHz, about 165 kHz, about 170 kHz, about 175 kHz, about 180 kHz, about 185 kHz, about 190 kHz, about 195 kHz, about 200 kHz, about 225 kHz, about 250 kHz, about 275 kHz, about 300 kHz, about 325 kHz, about 350 kHz, about 375 kHz, about 400 kHz, about 425 kHz, about 450 kHz, about 475 kHz, about 500 kHz, about 550 kHz, about 600 kHz, about 650 kHz, about 700 kHz, about 750 kHz, about 800 kHz, about 850 kHz, about 900 kHz, about 950 kHz, about 1 MHz, about 2 MHZ, about 3 MHz, about 4 MHZ, about 5 MHz, about 6 MHz, about 7 MHz, about 8 MHz, about 9 MHZ, about 10 MHZ, and the like, as well as a range formed from any of the above values (e.g., a range of from about 50 kHz to about 10 MHz, a range of from about 50 kHz to about 1 MHz, a range of from 50 kHz to about 500 kHz, a range of from about 100 kHz to about 500 kHz, a range of from about 150 kHz to about 300 kHz, etc.), and a range that combines two integers that fall between two of the above-referenced values (e.g., a range of from about 122 kHz to about 313 kHz, a range of from about 78 kHz to about 298 kHz, etc.).

In certain particular (but non-limiting) embodiments, the alternating electric field may be imposed at two or more different frequencies. When two or more frequencies are present, each frequency is selected from any of the above-referenced values, or a range formed from any of the above-referenced values, or a range that combines two integers that fall between two of the above-referenced values.

In certain particular (but non-limiting) embodiments, the following frequencies may be utilized for specific cancers: GBM, about 200 kHz; NSCLC, about 150 kHz; breast cancer, about 200 kHz; pancreatic cancer, about 150 kHz; brain metastases from NSCLC, about 150 kHz; hepatic cancer, about 150 kHz; and the like.

The alternating electric field may have any field strength in the target region/subject/cancer cells, so long as the alternating electric field is capable of functioning in accordance with the present disclosure. For example (but not by way of limitation), the alternating electric field may have a field strength in the target region/subject/cancer cells of at least about 1 V/cm, about 1.5 V/cm, about 2 V/cm, about 2.1 V/cm, about 2.2 V/cm, about 2.3 V/cm, about 2.4 V/cm, about 2.5 V/cm, about 2.6 V/cm, about 2.7 V/cm, about 2.8 V/cm, about 2.9 V/cm, about 2.5 V/cm, about 3 V/cm, about 3.5 V/cm, about 4 V/cm, about 4.5 V/cm, about 5 V/cm, about 5.5 V/cm, about 6 V/cm, about 6.5 V/cm, about 7 V/cm, about 7.5 V/cm, about 8 V/cm, about 9 V/cm, about 9.5 V/cm, about 10 V/cm, about 10.5 V/cm, about 11 V/cm, about 11.5 V/cm, about 12 V/cm, about 12.5 V/cm, about 13 V/cm, about 13.5 V/cm, about 14 V/cm, about 14.5 V/cm, about 15 V/cm, about 15.5 V/cm, about 16 V/cm, about 16.5 V/cm, about 17 V/cm, about 17.5 V/cm, about 18 V/cm, about 18.5 V/cm, about 19 V/cm, about 19.5 V/cm, about 20 V/cm, and the like, as well as a range formed from any of the above values (e.g., a range of from about 1 V/cm to about 20 V/cm, a range of from about 1 V/cm to about 10 V/cm, a range of from about 1 V/cm to about 4 V/cm, etc.), and a range that combines two integers that fall between two of the above-referenced values (e.g., a range of from about 1.1 V/cm to about 18.6 V/cm, a range of from about 1.2 V/cm to about 9.8 V/cm, a range of from about 1.3 V/cm to about 4.7 V/cm, etc.). Generally, it is desired to utilize the highest field strength possible without causing overheating, with field intensity typically being capped by temperature measurements.

In some instances, the electric field in at least a portion of the target region/subject/cancer cells is induced by an applied voltage that is determined by computer simulation of the target region/subject/cancer cells. In some instances, the electric field in at least a portion of the target region/subject/cancer cells is induced by an applied voltage of at least 50 V RMS (root mean squared) or at least 50 V p2p (peak-to-peak), and optionally, the applied voltage is at least 100 V RMS or at least 100 V p2p. In some embodiments, an applied voltage of at least 50 V induces an electric field with a field strength of at least 1 V/cm (e.g., at least 5 V/cm) in at least a portion of the target region/subject/cancer cells.

The alternating electric field may be applied in a single direction between a pair of arrays or may be alternating in two or more directions/channels between two or more pairs of arrays (e.g., front-back and left-right). For example, certain TTFields devices (such as, but not limited to, the OPTUNE® system (Novocure Limited, St. Helier, Jersey)) operate in two directions in order to increase the chances that a dividing cell will be aligned with the electric field such that the electric field can have the desired anti-mitotic effect. However, it will be understood that the scope of the present disclosure also includes the application of the alternating electric field in a single direction. The term “alternating electric field” as used herein will be understood to include application in a single direction/channel as well as in two or more directions/channels; in addition, the term “alternating electric field” as used herein will be understood to include both application of a single alternating electric field as well as application of a plurality of alternating electric fields in succession for a duration of time.

The alternating electric field may be applied for any continuous or cumulative period of time sufficient to achieve an increase in reactive oxygen species (ROS) levels and/or an enhancement in the cytotoxicity of the thioredoxin reductase/glutathione inhibitor(s), a reduction in viability of cancer cells, a reduction in tumor volume, and/or a prevention of increase in tumor volume. The period of time that the alternating electric field is applied includes both a continuous period of time as well as a cumulative period of time. That is, the period of time that the alternating electric field is applied includes a single session (i.e., continuous application) as well as multiple sessions with minor breaks in between sessions (i.e., consecutive application for a cumulative period). For example, a subject is allowed to take breaks during treatment with an alternating electric field device and is only expected to have the device positioned on the body and operational for at least about 60%, at least about 70%, or at least about 80% of the total treatment period (e.g., over a course of one day, one week, two weeks, one month, two months, three months, four months, five months, etc.).

For example, but not by way of limitation, the alternating electric field may be applied for a continuous or cumulative period of time of at least about 1 hour, about 2 hours, about 3 hours, about 4 hours, about 5 hours, about 6 hours, about 7 hours, about 8 hours, about 9 hours, about 10 hours, about 11 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 21 days, about 1 month, about 2 months, about 3 months, about 4 months, about 5 months, about 6 months, and the like, as well as a range formed from any of the above values (e.g., a range of from about 1 hour to about six months, a range of from about 1 hour to about 7 days, a range of from about 24 hours to about 72 hours, etc.), and a range that combines two integers that fall between two of the above-referenced values (e.g., a range of from about 14 hours to about 68 hours, etc.).

In a particular (but non-limiting) embodiment, the period of time that the alternating electric field is applied is at least about 24 hours, with the device positioned on the body and operational for at least about 80% of that period.

The total period of time that the alternating electric field is applied may be achieved in a continuous or intermittent manner. That is, when the alternating electric field is applied for a shorter period of time (such as, but not limited to, less than about 12 or 24 hours), the alternating electric field may be continuously applied over that period of time. However, when the alternating electric field is applied for a longer period of time (such as, but not limited to, a period of about 24 hours or greater), the treatment period may include one or more breaks during the application cycle that separate two or more application sections, whereby the application sections and breaks combine to form the total application period. When breaks are present, the alternating electric field is applied for at least about 50%, about 60%, about 70%, about 80%, or about 90% or more of the treatment time, so that the breaks typically constitute only about 10%, about 20%, about 30%, about 40%, about 50%, or less of the treatment time. In a particular (but non-limiting) embodiment, when breaks are present, the breaks should typically constitute about 20% or less of the treatment time, so that the alternating electric field is applied for at least about 80% or more of the treatment time. For example, but not by way of limitation, the alternating electric field should be applied for at least about 19 hours of each 24-hour period. In addition, the longer that the alternating electric field is applied, the higher the efficacy will be.

Any thioredoxin reductase inhibitor known in the art or otherwise contemplated herein may be utilized in accordance with the present disclosure, so long as the thioredoxin reductase inhibitor is capable of concurrent therapy with alternating electric fields to increase the ROS levels in cancer cells, reduce the viability of cancer cells, reduce the volume of a tumor, and/or prevent an increase in a volume of a tumor. In certain particular (but non-limiting) embodiments, the thioredoxin reductase inhibitor is an inhibitor of TrxR1 and/or TrxR2. Non-limiting examples of thioredoxin reductase inhibitors that can be utilized in accordance with the present disclosure include Auranofin (RIDAURA®, Prometheus Laboratories Inc., San Diego, CA), Ethaselen ((1,2-[bis(1,2-benzisoselenazolone-3 (2H)-ketone)]ethane; also known as BBSKE (Ye et al. (2017) J Zhejiang Univ Sci B. 18 (5): 373-382)), PX-12, PMX 464, chaetocin, TXNIP-IN-1, TrxR inhibitor D9, TRi-1, TrxR-IN-2, TrxR-IN-5, DVD-445, aurothioglucose, MitoCur-1, ZC0109, CS3, MJ25, gold (I) N-heterocyclic carbene (MC3), RITA (NSC 652287), piperlongumine, and the like, as well as any combinations thereof.

Any glutathione inhibitor known in the art or otherwise contemplated herein may be utilized in accordance with the present disclosure, so long as the glutathione inhibitor is capable of concurrent therapy with alternating electric fields to increase the ROS levels in cancer cells, reduce the viability of cancer cells, reduce the volume of a tumor, and/or prevent an increase in a volume of a tumor. Non-limiting examples of glutathione inhibitors that can be utilized in accordance with the present disclosure include erastin, buthionine sulfoximine (BSO), sulfasalazine, BPTES B-839, BSO sulfasalazine, PRLX 93936, Glutathione synthesis-IN-1 (DC-1), GPX4-IN-3, ML-162, ML-210, S-(p-Nitrobenzyl) glutathione, S-(2,4-Dinitrophenyl) glutathione, S-Hexylglutathione, Senecionine, JKE-1674, decylubiquinone, S-Octylglutathione, GSTO1-IN-1, GSTO1-IN-2, S-Benzylglutathione, coniferyl ferulate, curzerene, LCS3, RSL3, Ethacrynic acid, NBDHEX, PKUMDL-LC-101-D04, DL-Buthionine-Sulfoximine (BSO, DL-Buthionine-(S,R)-sulfoximine), and the like, as well as any combinations thereof.

The term “thioredoxin reductase/glutathione inhibitor(s)” as used herein refers to at least one thioredoxin reductase inhibitor, at least one glutathione inhibitor, and/or a combination of at least one thioredoxin reductase inhibitor and at least one glutathione inhibitor.

In certain non-limiting embodiments, the composition may comprise at least one thioredoxin reductase inhibitor or at least one glutathione inhibitor. In other particular (but non-limiting) embodiments, the composition may comprise both a thioredoxin reductase inhibitor and a glutathione inhibitor. For example (but not by way of limitation), the combination of the thioredoxin reductase inhibitor piperlongumine with the glutathione inhibitor erastin has been shown to possess a synergistic effect over either treatment alone when treating cancer cells (Yang et al. (2022) Antioxidants, 11 (4): 710). Therefore, the scope of the present disclosure explicitly includes combinations of any of the above-listed non-limiting examples of thioredoxin reductase inhibitors with any of the above-listed non-limiting examples of glutathione inhibitors.

The composition comprising at least one thioredoxin reductase/glutathione inhibitor may be provided with any formulation known in the art or otherwise contemplated herein. In certain particular (but non-limiting) embodiments, the composition comprising thioredoxin reductase/glutathione inhibitor(s) contains one or more pharmaceutically acceptable carriers (and as such, the composition may also be referred to as a “pharmaceutical composition”). Non-limiting examples of suitable pharmaceutically acceptable carriers that may be utilized in accordance with the present disclosure include water; saline; dextrose solutions; fructose or mannitol; calcium carbonate; cellulose; ethanol; oils of animal, vegetative, or synthetic origin; carbohydrates, such as glucose, sucrose, or dextrans; antioxidants, such as ascorbic acid or glutathione; chelating agents; low molecular weight proteins; detergents; liposomal carriers; conductive and non-conductive nanoparticles; buffered solutions, such as sodium chloride, saline, phosphate-buffered saline, and/or other substances which are physiologically acceptable and/or safe for use; diluents; excipients such as polyethylene glycol (PEG); or any combination thereof. Suitable pharmaceutically acceptable carriers for pharmaceutical formulations are described, for example, in Remington: The Science and Practice of Pharmacy, 23rd ed. (2020).

In certain particular (but non-limiting) embodiments, the composition comprising thioredoxin reductase/glutathione inhibitor(s) may further contain one or more additional active agents. Various active agents that can be utilized concurrently with alternating electric fields and/or thioredoxin reductase/glutathione inhibitor(s) are known in the art, and certain combination therapies are approved by the FDA or currently in clinical trials testing. Non-limiting examples of therapeutic agents that can be utilized in accordance with the present disclosure include anti-PD-1 therapeutics such as (but not limited to) Pembrolizumab (KEYTRUDA®, Merck & Co., Inc., Rahway, NJ), Tislelizumab, Nivolumab, and Cemiplimab; anti-PD-L1 therapeutics such as atezolizumab, avelumab, and durvalumab; chemotherapeutic agents, such as (but not limited to) Lenvatinib, Paclitaxel, Doxorubicin, Cisplatin, sorafenib, Docetaxel, Ifosamide, Etoposide (VEPESID®, Bristol-Myers Squibb Co, New York, NY), Gemcitabine, Lomustine, Nab Paclitaxel, Temozolomide, and Carboplatin; immune checkpoint inhibitors, such as (but not limited to) Cemiplimab, Nivolumab, Pembrolizumab, and Tislelizumab; TKI inhibitors, such as (but not limited to) lenvatinib and everolimus; mTOR inhibitors; Akt inhibitors; PI3K inhibitors; PARP inhibitors; VEGF inhibitors; FGF inhibitors; anti-LAB3 agents; aromatase inhibitors, such as (but not limited to) Letrozole; biologics, such as monoclonal antibodies (such as, but not limited to, Denosumab and pembrolizumab)); anti-LAG3 agents, such as (but not limited to) OPDUALAG™ and Relatimab; anti-CTLA-4 therapeutic agents, such as (but not limited to) Ipilimumab; and the like, as well as any combinations thereof.

In certain particular (but non-limiting) embodiments, the thioredoxin reductase/glutathione inhibitor(s) present in the composition is conjugated to another substance. For example, but not by way of limitation, the thioredoxin reductase/glutathione inhibitor(s) may be conjugated to a particle or other substance for targeted delivery of the drug to a specific location in the body. In another particular (but non-limiting) embodiment, the composition may comprise at least one thioredoxin reductase and/or at least one glutathione inhibitor encapsulated in a nanoparticle for phototherapy.

In addition, any of the compositions of the present disclosure may contain other agents that allow for administration of the compositions via a particular administration route. For example, but not by way of limitation, the compositions may be formulated for administration by oral, topical, transdermal, parenteral, subcutaneous, intranasal, mucosal, intramuscular, intraperitoneal, intravitreal, and/or intravenous routes. Based on the route of administration, the compositions may also contain one or more additional components in addition to the active agent(s) (e.g., thioredoxin reductase/glutathione inhibitor and/or additional therapeutic agent). Examples of additional secondary compounds that may be present include, but are not limited to, fillers, gels, adhesives, salts, buffers, preservatives, stabilizers, solubilizers, wetting agents, emulsifying agents, dispersing agents, and other materials well known in the art.

In particular (but non-limiting) embodiments, the at least one composition comprising at least one thioredoxin reductase/glutathione inhibitor is administered orally, intradermally, subcutaneously, intravenously, and/or intranodally to the cells/subject/tumor.

The at least one composition comprising at least one thioredoxin reductase inhibitor and/or at least one glutathione inhibitor may be administered before or after application of the alternating electric field has begun. In certain particular (but non-limiting) embodiments, the at least one composition comprising at least one thioredoxin reductase/glutathione inhibitor may be administered after the application of the alternating electric field has begun. In particular (but not by way of limitation), the at least one composition comprising at least one thioredoxin reductase/glutathione inhibitor may be administered during application of the alternating electric field (e.g., before the period of time that the alternating electric field is applied has elapsed) and/or after application of the alternating electric field has elapsed.

For example (but not by way of limitation), the at least one composition comprising at least one thioredoxin reductase inhibitor and/or at least one glutathione inhibitor may be administered before application of the alternating electric field has commenced by a period of at least about 1 minute, about 5 minutes, about 10 minutes, about 15 minutes, about 30 minutes, about 45 minutes, about 1 hour, about 1.5 hours, about 2 hours, about 3 hours, about 4 hours, about 5 hours, about 6 hours, about 7 hours, about 8 hours, about 9 hours, about 10 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 5 days, about 6 days, about 7 days, and the like, as well as a range formed from any of the above values (e.g., a range of from about 1 minute to about 24 hours, a range of from about 24 hours to about 96 hours, etc.), and a range that combines two integers that fall between two of the above-referenced values (e.g., a range of from about 14 hours to about 94 hours, etc.). In a particular (but non-limiting) embodiment, the at least one composition comprising at least one thioredoxin reductase inhibitor and/or at least one glutathione inhibitor is administered at least about 24 hours before application of the alternating electric field has begun.

In other non-limiting examples, the at least one composition comprising at least one thioredoxin reductase inhibitor and/or at least one glutathione inhibitor may be administered after application of the alternating electric field has commenced by a period of at least about 1 minute, about 5 minutes, about 10 minutes, about 15 minutes, about 30 minutes, about 45 minutes, about 1 hour, about 1.5 hours, about 2 hours, about 3 hours, about 4 hours, about 5 hours, about 6 hours, about 7 hours, about 8 hours, about 9 hours, about 10 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 5 days, about 6 days, about 7 days, and the like, as well as a range formed from any of the above values (e.g., a range of from about 1 minute to about 24 hours, a range of from about 24 hours to about 96 hours, etc.), and a range that combines two integers that fall between two of the above-referenced values (e.g., a range of from about 14 hours to about 94 hours, etc.). In a particular (but non-limiting) embodiment, the at least one composition comprising at least one thioredoxin reductase inhibitor and/or at least one glutathione inhibitor is administered at least about 24 hours after application of the alternating electric field has begun.

In yet other non-limiting examples, the at least one composition comprising at least one thioredoxin reductase inhibitor and/or at least one glutathione inhibitor may be administered after the period of time that the alternating electric field is applied has elapsed, wherein the at least one composition comprising at least one thioredoxin reductase/glutathione inhibitor is administered within about 1 minute, about 5 minutes, about 10 minutes, about 15 minutes, about 30 minutes, about 45 minutes, about 1 hour, about 1.5 hours, about 2 hours, about 3 hours, about 4 hours, about 5 hours, about 6 hours, about 7 hours, about 8 hours, about 9 hours, about 10 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, and the like, of when the period of time elapsed.

In a particular (but non-limiting) embodiment, the at least one composition comprising at least one thioredoxin reductase inhibitor and/or at least one glutathione inhibitor is administered within about 96 hours of when the period of time elapsed.

The composition comprising at least one thioredoxin reductase inhibitor and/or at least one glutathione inhibitor may be administered to the cancer cells/subject at any concentration that provides a therapeutically effective concentration of the at least one thioredoxin reductase/glutathione inhibitor. In certain non-limiting embodiments, the application of the alternating electric field reduces the amount of thioredoxin reductase/glutathione inhibitor(s) required to be therapeutically effective when compared to a normal therapeutically effective amount administered in the absence of an alternating electric field. For example, but not by way of limitation, the therapeutically effective concentration of thioredoxin reductase/glutathione inhibitor(s) may be reduced by at least about 25%, about 30%, about 35%, about 40%, about 45%, about 50%, about 55%, about 60%, about 65%, about 70%, about 75% or more with respect to a dosage of thioredoxin reductase/glutathione inhibitor known to be therapeutically effective in the absence of application of an alternating electric field. In a particular (but non-limiting) embodiment, the therapeutically effective concentration of thioredoxin reductase/glutathione inhibitor(s) is reduced by at least about 50% when compared to a dosage of thioredoxin reductase/glutathione inhibitor known to be therapeutically effective in the absence of an alternating electric field.

The therapeutically effective concentration of each thioredoxin reductase/glutathione inhibitor(s) utilized in accordance with the present disclosure may be, for example (but not by way of limitation), about 1 nM, about 10 nM, about 20 nM, about 30 nM, about 40 nM, about 50 nM, about 60 nM, about 70 nM, about 80 nM, about 90 nM, about 100 nM, about 125 nM, about 150 nM, about 175 nM, about 200 nM, about 250 nM, about 300 nM, about 350 nM, about 400 nM, about 450 nM, about 500 nM, about 550 nM, about 600 nM, about 650 nM, about 700 nM, about 750 nM, about 800 nM, about 850 nM, about 900 nM, about 950 nM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 25 mM, about 30 mM, about 35 mM, about 40 mM, about 45 mM, about 50 mM, and the like, as well as a range formed from any of the above values (e.g., a range of from about 12.5 nM to about 100 nM, a range of from about 1 mM to about 20 mM, etc.), and a range that combines two integers that fall between two of the above-referenced values (e.g., a range of from about 17 nM to about 83 nM, etc.).

In a particular (but non-limiting) embodiment, the therapeutically effective concentration of each thioredoxin reductase/glutathione inhibitor is from about 12.5 nM to about 100 nM.

In particular (but non-limiting) embodiments, the therapeutically effective concentration of each thioredoxin reductase/glutathione inhibitor utilized in accordance with the present disclosure may be, for example (but not by way of limitation), about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, and the like, as well as a range formed from any of the above values (e.g., a range of from about 10 mg/kg to about 50 mg/kg, a range of from about 1 mg/kg to about 40 mg/kg, a range of from about 1 mg/kg to about 30 mg/kg, a range of from about 1 mg/kg to about 25 mg/kg, a range of from about 1 mg/kg to about 20 mg/kg, a range of from about 1 mg/kg to about 10 mg/kg, etc.).

In certain particular (but non-limiting) embodiments, the thioredoxin reductase/glutathione inhibitor may be administered by any dosage regimen known in the art. For example, but not by way of limitation, the composition comprising the thioredoxin reductase/glutathione inhibitor may be administered in a single dosage or multiple dosages over a defined treatment period. For example (but not by way of limitation), a therapeutically effective concentration of the composition may be administered about once every 4 hours, about once every 8 hours, about once every 12 hours, about once every day, about once every other day, about once every three days, about once a week, about twice a week, about three times a week, about once every two weeks, about once every three weeks, about once a month, and the like, as well as a range formed from any of the above values (a range of about once every 4 to 8 hours, a range of from about once a week to about once a month, etc.).

In certain particular (but non-limiting) embodiments, the method includes one or more additional steps. For example (but not by way of limitation), the method may further include the step of (3) discontinuing the application of the alternating electric field (such as, but not limited to) to allow the cells/tissue to recover. In addition, any of steps (1) and/or (2) may be repeated one or more times.

In certain particular (but non-limiting) embodiments, the method involves concurrent therapy with two or more compositions. As such, the method may include an additional step of (4) administering at least a second composition to the cancer cells/subject. In one particular (but non-limiting) embodiment, both of the at least one thioredoxin reductase inhibitor and the at least one glutathione inhibitor are administered to the cancer cells/subject in separate compositions (i.e., the first composition comprises at least one thioredoxin reductase inhibitor, and the second composition comprises at least one glutathione inhibitor, or vice versa). Alternatively, and/or in addition thereto, the at least second composition may contain one or more of any of the active substances disclosed or otherwise contemplated herein for use with thioredoxin reductase/glutathione inhibitor(s). In yet a further alternative, at least three compositions may be administered to the cancer cells/subject, with the first composition comprising at least one thioredoxin reductase inhibitor and the second composition comprising at least one glutathione inhibitor (or vice versa), and then a third composition is administered to the cancer cells/subject that contains one or more of any of the active substances disclosed or otherwise contemplated herein for use with thioredoxin reductase/glutathione inhibitor(s).

Various substances and therapies that can be utilized in combination with thioredoxin reductase/glutathione inhibitor(s) are known in the art or otherwise contemplated herein. Non-limiting examples of therapeutic agents that can be present in the second composition (or third composition, when both thioredoxin reductase and glutathione inhibitors are administered and are present in the first and second compositions) and utilized in combination with thioredoxin reductase/glutathione inhibitor(s) in accordance with the present disclosure include anti-PD-1 therapeutics such as (but not limited to) Pembrolizumab (KEYTRUDA®, Merck & Co., Inc., Rahway, NJ), Tislelizumab, Nivolumab, and Cemiplimab; anti-PD-L1 therapeutics such as atezolizumab, avelumab, and durvalumab; chemotherapeutic agents, such as (but not limited to) Lenvatinib, Paclitaxel, Doxorubicin, Cisplatin, sorafenib, Docetaxel, Ifosamide, Etoposide (VEPESID®, Bristol-Myers Squibb Co, New York, NY), Gemcitabine, Lomustine, Nab Paclitaxel, Temozolomide, and Carboplatin; immune checkpoint inhibitors, such as (but not limited to) Cemiplimab, Nivolumab, Pembrolizumab, and Tislelizumab; TKI inhibitors, such as (but not limited to) lenvatinib and everolimus; mTOR inhibitors; Akt inhibitors; PI3K inhibitors; PARP inhibitors; VEGF inhibitors; FGF inhibitors; anti-LAB3 agents; anti-CTLA-4 therapeutics; aromatase inhibitors, such as (but not limited to) Letrozole; biologics, such as monoclonal antibodies (such as, but not limited to, Denosumab and pembrolizumab)); anti-LAG3 agents, such as (but not limited to) OPDUALAG™ and Relatimab; anti-PD-L1 therapeutic agents, such as (but not limited to) Atezolizumab, Avelumab, and Durvalumab; anti-CTLA-4 therapeutic agents, such as (but not limited to) Ipilimumab; and the like, as well as any combinations thereof.

When present, step (4) may be performed substantially simultaneously or wholly or partially sequentially with the administration of the first composition in step (1), whereby the two (or three) separate compositions are administered simultaneously or wholly or partially sequentially. In addition, the two (or three) compositions administered in steps (1) and (4) may be administered via the same route (e.g., both orally administered or injected), or the two (or three) compositions may be administered by different routes (e.g., one composition orally administered and another composition intravenously administered, etc.).

When present, the optional additional administration step (4) may be performed before or after the application of the alternating electric field has begun, and during application of the alternating electric field and/or after application of the alternating electric field has elapsed, in the same manner(s) and time frame(s) as described above for the first composition.

That is, for example (but not by way of limitation), the second composition (and/or third composition, when present) may be administered before application of the alternating electric field has commenced by a period of at least about 1 minute, about 5 minutes, about 10 minutes, about 15 minutes, about 30 minutes, about 45 minutes, about 1 hour, about 1.5 hours, about 2 hours, about 3 hours, about 4 hours, about 5 hours, about 6 hours, about 7 hours, about 8 hours, about 9 hours, about 10 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 5 days, about 6 days, about 7 days, and the like, as well as a range formed from any of the above values (e.g., a range of from about 1 minute to about 24 hours, a range of from about 24 hours to about 96 hours, etc.), and a range that combines two integers that fall between two of the above-referenced values (e.g., a range of from about 14 hours to about 94 hours, etc.). In a particular (but non-limiting) embodiment, the second (and/or third) composition(s) is/are administered at least about 24 hours before application of the alternating electric field has begun.

In other non-limiting examples, the second composition (and/or third composition, when present) may be administered after application of the alternating electric field has commenced by a period of at least about 1 minute, about 5 minutes, about 10 minutes, about 15 minutes, about 30 minutes, about 45 minutes, about 1 hour, about 1.5 hours, about 2 hours, about 3 hours, about 4 hours, about 5 hours, about 6 hours, about 7 hours, about 8 hours, about 9 hours, about 10 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 5 days, about 6 days, about 7 days, and the like, as well as a range formed from any of the above values (e.g., a range of from about 1 minute to about 24 hours, a range of from about 24 hours to about 96 hours, etc.), and a range that combines two integers that fall between two of the above-referenced values (e.g., a range of from about 14 hours to about 94 hours, etc.). In a particular (but non-limiting) embodiment, the second (and/or third) composition(s) is/are administered at least about 24 hours after application of the alternating electric field has begun.

In yet other non-limiting examples, the second composition (and/or third composition, when present) may be administered after the period of time that the alternating electric field is applied has elapsed, wherein the second (and/or third) composition(s) is/are administered within about 1 minute, about 5 minutes, about 10 minutes, about 15 minutes, about 30 minutes, about 45 minutes, about 1 hour, about 1.5 hours, about 2 hours, about 3 hours, about 4 hours, about 5 hours, about 6 hours, about 7 hours, about 8 hours, about 9 hours, about 10 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 5 days, about 6 days, about 7 days, and the like, of when the period of time elapsed.

In addition, for example (but not by way of limitation), the second composition (and/or third composition, when present) may be administered after administration of the first composition by a period of at least about 1 minute, about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 5 days, about 6 days, about 7 days, and the like, as well as a range formed from any of the above values (e.g., a range of from about 24 hours to about 96 hours, etc.), and a range that combines two integers that fall between two of the above-referenced values (e.g., a range of from about 14 hours to about 94 hours, etc.). In a particular (but non-limiting) embodiment, the second (and/or third) compositions is/are administered at least about 12 hours after administration of the first composition.

In certain particular (but non-limiting) embodiments, the method may further comprise the step of (5) administering at least one additional therapy to the cells/subject. Any therapies known in the art or otherwise contemplated herein for use with alternating electric fields (e.g., TTFields) and/or thioredoxin reductase/glutathione inhibitor therapy may be utilized in accordance with the methods of the present disclosure. Non-limiting examples of additional therapies that may be utilized include radiation therapy (such as, but not limited to, ionizing radiation therapy), photodynamic therapy, transarterial chemoembolization (TACE), or combinations thereof.

In certain particular (but non-limiting) embodiments, the method includes one or more additional steps. For example (but not by way of limitation), the method may further include repeating any of the steps (e.g., steps (1) and (2) and optional steps (3), (4), and (5)) one or more times. Each of the steps can be repeated as many times as necessary. When step (2) of applying the alternating electric field is repeated, the transducer arrays may be placed in slightly different positions on the subject than their original placement; relocation of the arrays in this manner may further aid in treatment of the tumor/cancer. In addition, step (1) and optional steps (4) and (5) (when present) of administering compositions/additional therapies may be repeated various times and at various intervals to follow any known and/or generally accepted dosage/treatment regimen for the composition(s)/therapy(ies).

The use of ordinal references to the optional steps is for purpose of example only; the methods of the present disclosure may include one or more of the optional steps (3), (4), and (5), either alone or in combination with one another. That is, the methods of the present disclosure include performing step (3) in the absence of steps (4) or (5), performing step (4) in the absence of steps (3) or (5), and performing step (5) in the absence of steps (3) and (4). In other words, the scope of the methods disclosed herein includes performing steps (1)-(2) (as well as repeating each step as many times as necessary), performing steps (1)-(3) (as well as repeating one or more steps as many times as necessary), performing steps (1)-(2) and (4) (as well as repeating one or more steps as many times as necessary), performing steps (1)-(2) and (5) (as well as repeating one or more steps as many times as necessary), performing steps (1)-(4) (as well as repeating one or more steps as many times as necessary), performing steps (1)-(3) and (5) (as well as repeating one or more steps as many times as necessary), performing steps (1)-(2) and (4)-(5) (as well as repeating one or more steps as many times as necessary), and performing all of steps (1)-(5) (as well as repeating one or more steps as many times as necessary).

While the use of concurrent therapy with two substances is explicitly described above, it will be understood that the scope of the present disclosure further includes concurrent therapy with three or more compositions. As such, the method can include one or more additional steps of administering an additional composition to the subject (similar to steps (1) and (4)). Any additional substances administered in the method may be selected from any of the substances disclosed or otherwise contemplated herein for use concurrently with thioredoxin reductase inhibitor(s) and/or glutathione inhibitor(s) (as disclosed herein above with respect to optional step (4)). Also, administration of any additional substances can be performed substantially simultaneously or wholly or partially sequentially with the administration of the first and/or second compositions/substances and in the same manner(s) and time frame(s) as described above for the first and second compositions/substances.

While the methods described herein above are related to use of the combination of alternating electric fields (e.g., TTFields) with at least one of thioredoxin reductase inhibitor and/or at least one glutathione inhibitor in cancer treatment, it will be understood that the scope of the present disclosure is not limited to use in cancer treatment. Rather, the present disclosure encompasses treatment of any other related diseases, infections, or conditions for which thioredoxin reductase/glutathione inhibitor treatment and/or alternating electric field treatment is beneficial.

Certain non-limiting embodiments of the present disclosure are related to kits that include any of the components of the alternating electric field-generating systems disclosed or otherwise contemplated herein (such as, but not limited to, one or more transducer arrays and/or one or more hydrogel compositions, as disclosed in U.S. Pat. Nos. 7,016,725; 7,089,054; 7,333,852; 7,565,205; 8,244,345; 8,715,203; 8,764,675; 10,188,851; and 10,441,776; and in US Patent Application Nos. US 2018/0160933; US 2019/0117956; US 2019/0307781; and US 2019/0308016) in combination with at least one of any of the compositions comprising thioredoxin reductase inhibitor(s) and/or glutathione inhibitor(s) disclosed or otherwise contemplated herein (including two separate compositions that include a thioredoxin reductase inhibitor(s)-containing composition and a glutathione inhibitor(s)-containing composition). The kits may optionally further include one or more of any of the optional compositions disclosed or otherwise contemplated herein (such as, but not limited to, one or more compositions utilized in one or more optional concurrent therapy step(s)). The kits may optionally further include one or more devices (or one or more components of devices) utilized in one or more additional therapy steps.

In a particular (but non-limiting) embodiment, the kit may further include instructions for performing any of the methods disclosed or otherwise contemplated herein. For example (but not by way of limitation), the kit may include instructions for applying one or more components of the alternating electric field-generating device to the skin of the patient, instructions for applying the alternating electric field to the patient, instructions for when and how to administer the composition(s) comprising at least one thioredoxin reductase inhibitor and/or at least one glutathione inhibitor and optionally how to administer one or more optional additional compositions, and/or instructions for when to activate and turn off the alternating electric field in relation to the administration of the composition(s) comprising at least one thioredoxin reductase inhibitor and/or at least one glutathione inhibitor and/or administration of one or more optional compositions.

In addition to the components described in detail herein above, the kits may further contain other component(s)/reagent(s) for performing any of the particular methods described or otherwise contemplated herein. For example (but not by way of limitation), the kits may additionally include: (i) components for preparing the skin prior to disposal of the hydrogel compositions and/or transducer arrays thereon (e.g., a razor, a cleansing composition or wipe/towel, etc.); (ii) components for removal of the gel/transducer array(s); (iii) components for cleansing of the skin after removal of the gel/transducer array(s); and/or (iv) other components utilized with the system (e.g., conductive material, nonconductive material, a soothing gel or cream, a bandage, etc.). The nature of these additional component(s)/reagent(s) will depend upon the particular treatment format, and identification thereof is well within the skill of one of ordinary skill in the art; therefore, no further description thereof is deemed necessary. Also, the components/reagents present in the kits may each be in separate containers/compartments, or various components/reagents can be combined in one or more containers/compartments, depending on the sterility, cross-reactivity, and stability of the components/reagents.

The kit may be disposed in any packaging that allows the components present therein to function in accordance with the present disclosure. In certain non-limiting embodiments, the kit further comprises a sealed packaging in which the components are disposed. In certain particular (but non-limiting) embodiments, the sealed packaging is substantially impermeable to air and/or substantially impermeable to light.

In addition, the kit can further include a set of written instructions explaining how to use one or more components of the kit. A kit of this nature can be used in any of the methods described or otherwise contemplated herein.

In certain non-limiting embodiments, the kit has a shelf life of at least about six months, such as (but not limited to), at least about nine months, or at least about 12 months.

Certain non-limiting embodiments of the present disclosure are related to systems that include any of the components of the alternating electric field-generating systems disclosed or otherwise contemplated herein (such as, but not limited to, one or more transducer arrays and/or one or more hydrogel compositions, as disclosed in U.S. Pat. Nos. 7,016,725; 7,089,054; 7,333,852; 7,565,205; 8,244,345; 8,715,203; 8,764,675; 10,188,851; and 10,441,776; and in US Patent Application Nos. US 2018/0160933; US 2019/0117956; US 2019/0307781; and US 2019/0308016) in combination with at least one of any of the compositions comprising at least one thioredoxin reductase inhibitor and/or at least one glutathione inhibitor disclosed or otherwise contemplated herein. The systems may optionally further include one or more of any of the optional compositions disclosed or otherwise contemplated herein. The systems may optionally further include one or more devices (or one or more components of devices) utilized in one or more additional therapy steps.

### EXAMPLES

Examples are provided hereinbelow. However, the present disclosure is to be understood to not be limited in its application to the specific experimentation, results, and laboratory procedures disclosed herein after. Rather, the Examples are simply provided as one of various embodiments and are meant to be exemplary, not exhaustive.

### Example 1

Typically, cancer cells exhibit higher basal levels of ROS compared with normal cells. In order to maintain cellular processes and cell proliferation, cancer cells have elevated antioxidant defenses, largely through upregulation of the Trx and GSH systems. The purpose of the present disclosure is to tilt the scale to achieve high ROS levels and increase cancer cell death through concurrent treatment of TTFields with thioredoxin reductase inhibitors and/or glutathione inhibitors.

As stated in the Background section, there are several publications which show that TTFields can induce ROS production. For example, Park et al. (incorporated supra) demonstrated that ROS levels increased after TTFields treatment in macrophages, and Jo et al. (incorporated supra) demonstrated increased ROS levels after TTFields treatment in GBM cells.

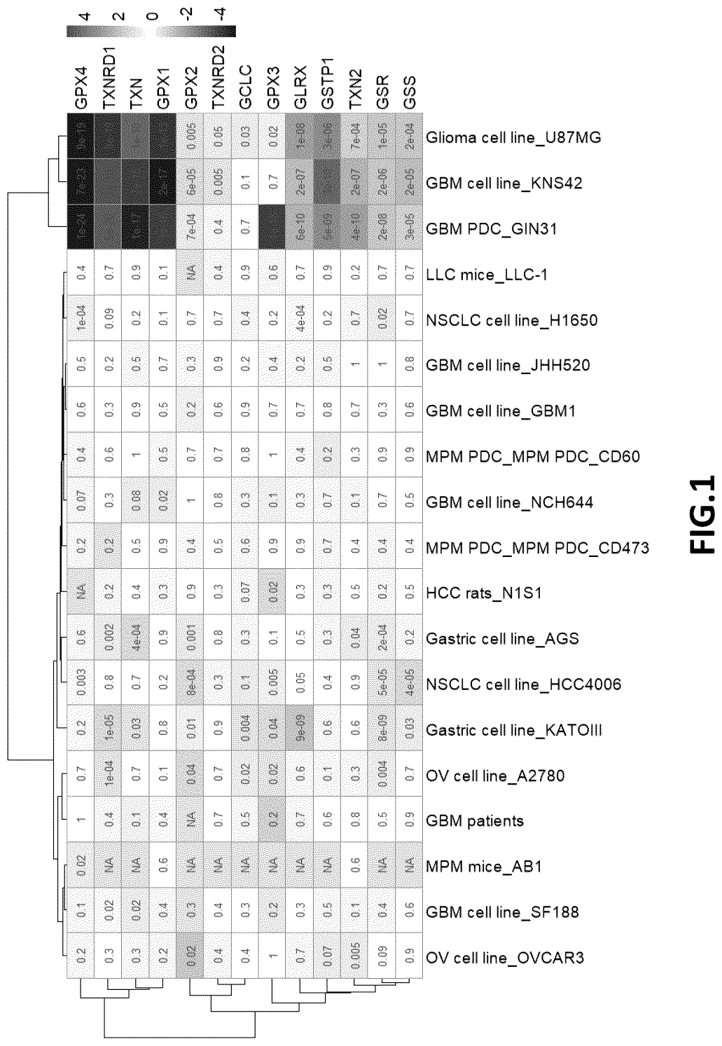

Therefore, the effects of TTFields treatment on the expression of various genes in the Trx and GSH pathways were examined through an analysis of RNA levels of various genes in several cancer cell lines following TTFields treatment. The data shown in FIG. 1 demonstrate a decrease in thioredoxin and glutathione related genes in response to TTFields treatment. These results indicate that there is a decrease in RNA synthesis for reducing proteins in response to TTFields. Thus, the ROS level increase in response to TTFields treatment observed in the prior art may be directly attributed to TTFields or indirectly due to the decrease in antioxidant defenses.

Based on the findings shown in FIG. 1, TTFields application is combined with the use of thioredoxin or glutathione inhibitors to further decrease the antioxidant defense in cells. Inhibition of thioredoxin or glutathione activity in accordance with the present disclosure pushes the cell towards producing more ROS that are not reduced, and therefore leads to cell death of cancer cells.

### Example 2

As described in Example 1, application of TTFields has been shown to induce reactive oxygen species (ROS) production. In this Example, the effects of concomitant treatment of TTFields with thioredoxin reductase inhibitor(s) and/or glutathione inhibitor(s) were studied; the concurrent treatment reduces the ability of cells to overcome oxidative stress, resulting in more cell death and higher efficacy of TTFields treatment.

GBM recurrence may result from: (1) DNA repair mechanisms, such as (but not limited to) methylation status of MGMT (O⁶-methylguanine-DNA methyl-transferase); (2) chemo- and radioresistant GBM stem cells; and/or (3) an adaptive response to oxidative stress. The thioredoxin (Trx) system plays an important role in the regulation of intracellular redox balance, and overexpression of thioredoxin reductase 1 (TrxR1) inhibits ROS increase and correlates with chemo/radioresistance and aggressive disease in GBM. TrxR1 is up-regulated in more than half of GBMs, and TrxR1 overexpression in GBM counteracts RT-induced ROS leading to radioresistance.

Auranofin (RIDAURA®, Prometheus Laboratories Inc., San Diego, CA) is an organogold compound used as a thioredoxin reductase (TrxR) inhibitor that interacts with TrxR and inhibits its activity, thereby leading to an increase in reactive oxygen species (ROS). Auranofin is FDA-approved for the treatment of Rheumatoid Arthritis. Auranofin is orally available, well-tolerated, and lipophilic, and Auranofin also crosses the blood-brain barrier. In accordance with the present disclosure, Auranofin can be utilized alone or in combination with one or more other agents. For example, Auranofin has also been tested in combination with eight other re-purposed older drugs in the CUSP-9 treatment protocol for recurrent glioblastoma.

In this Example, the efficacy of TTFields treatment is increased by combining TTFields treatment with auranofin treatment to inhibit the Trx system, thereby resulting in higher ROS levels, decreasing cell proliferation, and promoting cell death in cancer cells. The combination of TTFields with auranofin decreases protein levels of the Trx system, but also affects the protein levels of the GSH system, and thereby further elevates ROS, eventually leading to significant cell death.

In particular, in this Example, the combination of Auranofin and TTFields is used to produce a synergistic effect in glioblastoma stem cells (GSCs) and to overcome drug resistance in GBM. In addition, the combination of Auranofin and TTFields is further combined with radiation therapy (RT, also known as radiotherapy) to produce a synergistic effect in GSCs and overcome drug resistance in GBM.

In this Example, a functional and cellular assessment of trimodal therapy on ROS signaling, neurosphere formation, and cell growth in GSCs is performed. Preclinical testing of RT, AU, and TTFields is performed in vivo in GSCs.

### Research Design and Methods:

In vitro—To assess the effect of this trimodal therapy, GSCs are exposed to AU (auranofin, 0.50 μM), RT (3 Gy), TTFields, TTFields+AU, TTFields+RT, and the combination of all three treatments (AU+RT+TTFields). The GSCs are assessed for the following: (i) a neurosphere formation assay; (ii) ROS Intracellular levels; (iii) MitoTracker™ assessment of mitochondrial membrane potential; and (iv) TrXR1 and GSH activity assays.

In vivo—To further investigate the effect of ROS-inducing trimodal therapy (RT+AU+TTFields) in vivo, subcutaneous injections of GSCs 1123S and 528 stably transduced with luciferase are performed. GCSs are injected (3×10⁵ cells 10 μl) to the right flank of mice. Treatment is started when tumors can be visualized by BLI upon injection of D-luciferin substrate. Blood is collected to monitor potential toxicities. The tumor is exposed to snap-freezing and then processed for WB and for tissue formalin fixation for further IHC analysis of p53, MGMT, and TrxR1-associated genes.

### Example 3

A phase II randomized controlled trial is performed to study the combination of Auranofin, RT, and TTFields in recurrent GBM. The three-week treatment cycle includes the following treatments for patients in the trial: TTFields are administered daily during weeks 1, 2, and 3; Auranofin (3 mg dose) is administered orally twice a day during weeks 1, 2, and 3; radiation (dosage of 2 Gy/day) is administered for 5 daily fractions during week 3. This three-week cycle is then repeated until progression is observed.

### Example 4

In this Example, the combination of TTFields treatment with administration of a glutathione inhibitor is examined. Mice are treated with TTFields for 72 h using the INOVIVO™ system (Novocure GmbH, Root, Switzerland), followed by intraperitoneal injection of erastin at a dose of 15 mg/kg/day for three days at 24 h intervals.

The combination of TTFields treatment with administration of a glutathione inhibitor provides a synergistic effect over either treatment alone.

### Example 5

In this Example, various non-limiting examples of thioredoxin inhibitors and glutathione inhibitors that can be utilized in accordance with the present disclosure are provided.

TABLE 1
Non-limiting Examples of Thioredoxin Inhibitors

PX-12 (DB05448, 1-methyl propyl 2-imidazolyl disulfide) is a potent
thioredoxin-1 (Trx-1) inhibitor by irreversibly thioalkylation of
Cys73 of Trx-1. Phase 2.
PMX 464
Chaetocin is a specific inhibitor of the histone methyltransferase
(HMT) SU(VAR)3-9 with an IC₅₀ of 0.6 μM for SU(VAR)3-9. It also
inhibits thioredoxin reductase (TrxR) with an IC₅₀ of 4 μM.
TXNIP-IN-1 is TXNIP-TRX (thioredoxin-interacting protein-
thioredoxin) complex inhibitor
TrxR inhibitor D9 is a potent and selective inhibitor of thioredoxin
reductase (TrxR
TRi-1 is a potent, specific and irreversible inhibitor of cytosolic
thioredoxin reductase 1 (TXNRD1), with an IC₅₀ of 12 nM. TRi-1 has
little mitochondrial toxicity for anticancer therapy.
Ethaselen (BBSKE) is an orally active, selective thioredoxin
reductase (TrxR) inhibitor with IC₅₀s of 0.5 and 0.35 μM for the
wild-type human TrxR1 and rat TrxR1, respectively
TrxR-IN-2, a potential thioredoxin reductase (TrxR) inhibitor,
represents a promising candidate drug for the chemotherapy of
drug-resistant hepatocellular carcinoma.
DVD-445 (Compound 7) is a potent peptidomimetic covalent thioredoxin
reductase 1 (TrxR1) inhibitor
Aurothioglucose (Gold thioglucose), containing monovalent gold ion,
is a potent active-site inhibitor of TrxR1 (thioredoxin reductase 1)
MitoCur-1, a curcumin analogue, is an inhibitor of mitochondrial
antioxidative thioredoxin reductase 2 (TrxR2).
TrxR-IN-5 (compound 4f) is a potent TrxR (thioredoxin reductase)
inhibitor,
ZC0109 is a dual inhibitor of IDO1 and thioredoxin reductase 1 (TrxR1)

TABLE 2
Non-limiting Examples of Glutathione Inhibitors

Glutathione synthesis-IN-1 (DC-1) is a glutathione synthesis inhibitor.
GPX4-IN-3 (26a) is a potent glutathione peroxidase 4 (GPX4) inhibitor
as a selective ferroptosis inducer. GPX4-IN-3 (26a) exhibits 71.7%
inhibition for GPX4 with 1 μM.
ML 162- inhibits GPX4
S-(p-Nitrobenzyl)glutathione is a competitive glutathionase inhibitor.
S-(2,4-Dinitrophenyl)glutathione is a substrate for glutathione-S-
transferase. (2,4-Dinitrophenyl)glutathione can be used as an
irreversible glutathione reductase inhibitor with an Ki value of 30 μM.
S-Hexylglutathione is an S-substituted glutathione in which the hydrogen
of the thiol has been replaced by a hexyl group. S-Hexylglutathione is
also an competitive inhibitor against glutathione-S-transferase
ML-210 is a selective and covalent glutathione peroxidase 4 (GPX4)
inhibitor with an EC50 of 30 nM. ML-210 binds the GPX4 selenocysteine
residue. ML-210 has anti-cancer activity.
Senecionine (Senecionan-11,16-dione, 12-hydroxy-) is a pyrrolizidine
alkaloid could be isolated from Senecio vulgaris. Senecionine decreases
the activities of glutathione S-transferase, aminopyrine demethylase
and arylhydrocarbon hydroxylase (AHH).
JKE-1674 is an orally active glutathione peroxidase 4 (GPX4)
inhibitor and an active metabolite of GPX4 inhibitor ML-210.
Decylubiquinone is an analog of ubiquinone (coenzyme Q10).
Decylubiquinone blocks reactive oxygen species (ROS) production in
response to glutathione depletion and inhibits activation of the
mitochondrial permeability transition
S-Octylglutathione is a competitive glutathione S-transferase (GST)
inhibitor.
GSTO1-IN-1 is a potent glutathione S-transferase omega 1 (GSTO1)
inhibitor
GSTO-IN-2 is a glutathione S-transferase inhibitor
S-Benzylglutathione is a competitive glutathionase inhibitor.
Coniferyl ferulate, a strong inhibitor of glutathione S-transferase
(GST), reverses multidrug resistance and downregulates P-glycoprotein
Curzerene inhibits glutathione S-transferase A1 (GSTA1) mRNA and
protein expression
LCS3 is a reversible and uncompetitive glutathione disulfide reductase
(GSR) and thioredoxin reductase 1 (TXNRD1) inhibitor
RSL3 ((1S,3R)-RSL3) is an inhibitor of glutathione peroxidase 4 (GPX4) (
Ethacrynic acid is an inhibitor of glutathione S-transferases (GSTs)
NBDHEX is a potent glutathione S-transferase P1-1 (GSTP1-1) inhibitor
PKUMDL-LC-101-D04 (GPX4-Activator-1d4) is a potent ferroptosis
regulator glutathione peroxidase 4 (GPX4) allosteric activator
DL-Buthionine-Sulfoximine (BSO, DL-Buthionine-(S,R)-sulfoximine)
is an inhibitor of glutathione (GSH) synthesis.

### Non-Limiting Illustrative Embodiments of the Inventive Concept(s)

Illustrative embodiment 1. A method of reducing viability of cancer cells, the method comprising the steps of: (1) administering at least one composition to the cancer cells, wherein the at least one composition comprises at least one thioredoxin reductase inhibitor and/or at least one glutathione inhibitor; and (2) applying an alternating electric field to the cancer cells for a period of time. The method may be an in vitro method or an in vivo method.

Illustrative embodiment 2. A method of enhancing cytotoxicity of at least one thioredoxin reductase inhibitor and/or at least one glutathione inhibitor against cancer cells. The method includes the steps of: (1) administering at least one composition to the cancer cells, wherein the at least one composition comprises at least one thioredoxin reductase inhibitor and/or at least one glutathione inhibitor; and (2) applying an alternating electric field to the cancer cells for a period of time.

Illustrative embodiment 3. A method of increasing reactive oxygen species (ROS) in cancer cells. The method includes the steps of: (1) administering at least one composition to the cancer cells, wherein the at least one composition comprises at least one thioredoxin reductase inhibitor and/or at least one glutathione inhibitor; and (2) applying an alternating electric field to the cancer cells for a period of time.

Illustrative embodiment 4. The method of any of illustrative embodiments 1-3, wherein at least one of: the alternating electric field is applied at a frequency in a range of from about 50 kHz to about 1 MHz; the alternating electric field has a field strength of at least about 1 V/cm in at least a portion of the cancer cells; and the period of time that the alternating electric field is applied is at least about 50% of at least about a 24 consecutive hour time period.

Illustrative embodiment 5. The method of any of illustrative embodiments 1-4, wherein steps (1) and (2) are performed substantially simultaneously.

Illustrative embodiment 6. The method of any of illustrative embodiments 1-4, wherein steps (1) and (2) are performed wholly or partially sequentially, and wherein the at least one composition is administered before the application of the alternating electric field has begun.

Illustrative embodiment 7. The method of any of illustrative embodiments 1-4, wherein steps (1) and (2) are performed wholly or partially sequentially, and wherein the at least one composition is administered after the application of the alternating electric field has begun.

Illustrative embodiment 8. The method of any one of illustrative embodiments 1-4 and 7, wherein the at least one composition is administered before the period of time the alternating electric field is applied has elapsed.

Illustrative embodiment 9. The method of any one of illustrative embodiments 1-4 and 7, wherein the at least one composition is administered after the period of time has elapsed.

Illustrative embodiment 10. The method of any one of illustrative embodiments 1-9, wherein steps (1) and (2) are repeated one or more times.

Illustrative embodiment 11. The method of any one of illustrative embodiments 1-10, wherein the cancer cells are selected from the group consisting of hepatocellular carcinoma cells, glioblastoma cells, pleural mesothelioma cells, differentiated thyroid cancer cells, advanced renal cell carcinoma cells, ovarian cancers, breast cancers, pancreatic cancers, lung cancers, or combinations thereof.

Illustrative embodiment 12. A method of treating cancer in a subject, the method comprising the steps of: (1) administering at least one composition to the subject, wherein the at least one composition comprises at least one thioredoxin reductase inhibitor and/or at least one glutathione inhibitor; and (2) applying an alternating electric field to a target region of the subject.

Illustrative embodiment 13. A method of reducing a volume of a tumor, wherein the tumor is present in a body of a living subject and includes a plurality of cancer cells, the method comprising the steps of: (1) administering at least one composition to the subject, wherein the at least one composition comprises at least one thioredoxin reductase inhibitor and/or at least one glutathione inhibitor; and (2) applying an alternating electric field to a target region of the subject.

Illustrative embodiment 14. A method of preventing an increase in volume of a tumor, wherein the tumor is present in a body of a living subject and includes a plurality of cancer cells, the method comprising the steps of: (1) administering at least one composition to the subject, wherein the at least one composition comprises at least one thioredoxin reductase inhibitor and/or at least one glutathione inhibitor; and (2) applying an alternating electric field to a target region of the subject.

Illustrative embodiment 15. The method of any of illustrative embodiments 12-14, wherein the at least one composition is orally or intravenously administered to the subject.

Illustrative embodiment 16. The method of any of illustrative embodiments 12-15, wherein at least one of: the alternating electric field is applied at a frequency in a range of from about 50 kHz to about 1 MHz; the alternating electric field has a field strength of at least about 1 V/cm in at least a portion of the cancer cells; and the period of time that the alternating electric field is applied is at least about 50% of at least about a 24 consecutive hour time period.

Illustrative embodiment 17. The method of any one of illustrative embodiments 12-16, wherein steps (1) and (2) are performed substantially simultaneously.

Illustrative embodiment 18. The method of any one of illustrative embodiments 12-16, wherein steps (1) and (2) are performed wholly or partially sequentially, and wherein the at least one composition is administered before the application of the alternating electric field has begun.

Illustrative embodiment 19. The method of any one of illustrative embodiments 12-16, wherein steps (1) and (2) are performed wholly or partially sequentially, and wherein the at least one composition is administered after the application of the alternating electric field has begun.

Illustrative embodiment 20. The method of any one of illustrative embodiments 12-16 and 19, wherein the at least one composition is administered after the period of time has elapsed.

Illustrative embodiment 21. The method of any one of illustrative embodiments 12-16 and 19, wherein the at least one composition is administered before the period of time the alternating electric field is applied has elapsed.

Illustrative embodiment 22. The method of any one of illustrative embodiments 12-21, wherein steps (1) and (2) are repeated one or more times.

Illustrative embodiment 23. The method of any one of illustrative embodiments 12-22, wherein the cancer cells are selected from the group consisting of hepatocellular carcinoma cells, glioblastoma cells, pleural mesothelioma cells, differentiated thyroid cancer cells, advanced renal cell carcinoma cells, ovarian cancers, breast cancers, pancreatic cancers, lung cancers, and combinations thereof.

Illustrative embodiment 24. The method of any of illustrative embodiments 1-23, wherein the at least one thioredoxin reductase inhibitor is selected from the group consisting of auranofin, Ethaselen, PX-12, PMX 464, chaetocin, TXNIP-IN-1, TrxR inhibitor D9, TRi-1, TrxR-IN-2, TrxR-IN-5, DVD-445, aurothioglucose, MitoCur-1, ZC0109, CS3, MJ25, gold (I) N-heterocyclic carbene (MC3), RITA, piperlongumine, and combinations thereof.

Illustrative embodiment 25. The method of illustrative embodiment 24, wherein the at least one thioredoxin reductase inhibitor comprises auranofin.

Illustrative embodiment 26. The method of any of illustrative embodiments 1-25, wherein the at least one glutathione inhibitor is selected from the group consisting of erastin, buthionine sulfoximine (BSO), sulfasalazine, BPTES B-839, BSO sulfasalazine, PRLX 93936, Glutathione synthesis-IN-1 (DC-1), GPX4-IN-3, ML-162, ML-210, S-(p-Nitrobenzyl) glutathione, S-(2,4-Dinitrophenyl) glutathione, S-Hexylglutathione, Senecionine, JKE-1674, decylubiquinone, S-Octylglutathione, GSTO1-IN-1, GSTO1-IN-2, S-Benzylglutathione, coniferyl ferulate, curzerene, LCS3, RSL3, Ethacrynic acid, NBDHEX, PKUMDL-LC-101-D04, DL-Buthionine-Sulfoximine (BSO, DL-Buthionine-(S,R)-sulfoximine), and combinations thereof.

Illustrative embodiment 27. The method of any of illustrative embodiments 1-26, wherein the alternating electric field has a field strength in a range of from about 1 V/cm to about 10 V/cm in at least a portion of the cancer cells.

Illustrative embodiment 28. The method of any of illustrative embodiments 1-27, wherein the period of time that the alternating electric field is applied is in a range of from about 24 hours to about 72 hours.

Illustrative embodiment 29. The method of any of illustrative embodiments 1-28, wherein the method further comprises the step of discontinuing the application of the alternating electric field.

Illustrative embodiment 30. The method of any of illustrative embodiments 1-4, 7-16, and 19-29, wherein the at least one composition is administered at least 24 hours after application of the alternating electric field has begun.

Illustrative embodiment 31. The method of any of illustrative embodiments 1-4, 7, 9-16, 19-20, and 22-30, wherein the at least one composition is administered within about 96 hours of when the period of time elapsed.

Illustrative embodiment 32. The method of any of illustrative embodiments 1-31, wherein the at least one composition further comprises a pharmaceutically acceptable carrier.

Illustrative embodiment 33. The method of any of illustrative embodiments 1-32, wherein the composition comprising at least one thioredoxin reductase inhibitor and/or at least one glutathione inhibitor is administered to the cancer cells/subject at a therapeutically effective concentration of the at least one thioredoxin reductase/glutathione inhibitor.

Illustrative embodiment 34. The method of illustrative embodiment 33, wherein the therapeutically effective concentration of the at least one thioredoxin reductase/glutathione inhibitor is reduced by at least about 50% with respect to a dosage of the thioredoxin reductase/glutathione inhibitor known to be therapeutically effective in the absence of an alternating electric field.

Illustrative embodiment 35. The method of illustrative embodiment 34, wherein the therapeutically effective concentration of the at least one thioredoxin reductase/glutathione inhibitor is from about 10 mg/kg to about 50 mg/kg.

Illustrative embodiment 36. The method of any of illustrative embodiments 1-35, wherein the at least one composition further comprises at least one additional therapeutic agent.

Illustrative embodiment 37. The method of any of illustrative embodiments 1-36, wherein the method further comprises the step of administering a second composition to the cancer cells/subject.

Illustrative embodiment 38. The method of illustrative embodiment 37, wherein the first composition comprises at least one thioredoxin reductase inhibitor, and the second composition comprises at least one glutathione inhibitor, or vice versa, and wherein the first and second compositions are administered substantially simultaneously or wholly or partially sequentially.

Illustrative embodiment 39. The method of illustrative embodiment 37 or 38, wherein the second composition comprises at least one additional therapeutic agent, and wherein the first and second compositions are administered substantially simultaneously or wholly or partially sequentially.

Illustrative embodiment 40. The method of illustrative embodiment 36 or 39, wherein the at least one additional therapeutic agent is selected from the group consisting of a chemotherapeutic agent, an immune checkpoint inhibitor, an anti-PD-1 therapeutic agent, a TKI inhibitor, an mTOR inhibitor, an Akt inhibitor, a PI3K inhibitor, a PARP inhibitor, a VEGF inhibitor, an FGF inhibitor, an aromatase inhibitor, a radiotherapy agent, a biologic, an anti-LAG3 agent, an anti-PD-L1 therapeutic agent, an anti-CTLA-4 therapeutic agent, and combinations thereof.

Illustrative embodiment 41. The method of illustrative embodiment 40, wherein the at least one additional therapeutic agent is selected from the group consisting of Paclitaxel, Doxorubicin, Cisplatin, sorafenib, Docetaxel, Ifosamide, Etoposide (Vepesid), Gemcitabine, Lomustine, Nab Paclitaxel, Temozolomide, Carboplatin, Cemiplimab, Nivolumab, Pembrolizumab, Tislelizumab, lenvatinib, everolimus, Letrozole, Denosumab, Relatimab, Atezolizumab, Avelumab, Durvalumab, Ipilimumab, and combinations thereof.

Illustrative embodiment 42. The method of any of illustrative embodiments 1-41, further comprising the step of administering at least one additional therapy to the cancer cells/subject.

Illustrative embodiment 43. The method of illustrative embodiment 42, wherein the at least one additional therapy is selected from the group consisting of radiation therapy, photodynamic therapy, transarterial chemoembolization (TACE), and combinations thereof.

Illustrative embodiment 44. The method of any of illustrative embodiments 1-43, further comprising the step of exposing the cancer cells/subject to radiation therapy, wherein the radiation therapy is applied before step (1), after step (1), before step (2), and/or after step (2).

Illustrative embodiment 45. The method of any of illustrative embodiments 1-44, further comprising the step of exposing the cancer cells/target region of the subject to ionizing radiation (IR), wherein the IR is applied before step (1), after step (1), before step (2), and/or after step (2).

Illustrative embodiment 46. Use of at least one composition comprising at least one thioredoxin reductase inhibitor and/or at least one glutathione inhibitor in a concomitant therapy method of any of illustrative embodiments 1-45.

Illustrative embodiment 47. At least one composition comprising at least one thioredoxin reductase inhibitor and/or at least one glutathione inhibitor for use in any of the methods of illustrative embodiments 1-35.

While the attached disclosures describe the inventive concept(s) in conjunction with the specific experimentation, results, and language set forth hereinafter, it is evident that many alternatives, modifications, and variations will be apparent to those skilled in the art. Accordingly, it is intended to embrace all such alternatives, modifications, and variations that fall within the spirit and broad scope of the present disclosure.

1. A method of reducing viability of cancer cells, the method comprising the steps of:
(1) administering at least one composition to the cancer cells, wherein the at least one composition comprises at least one thioredoxin reductase inhibitor and/or at least one glutathione inhibitor; and
(2) applying an alternating electric field to the cancer cells for a period of time.

2. The method of claim 1, wherein at least one of:
the alternating electric field is applied at a frequency in a range of from about 50 kHz to about 1 MHz;
the alternating electric field has a field strength of at least about 1 V/cm in at least a portion of the cancer cells;
the period of time that the alternating electric field is applied is at least about 50% of at least about a 24 consecutive hour time period; and
steps (1) and (2) are repeated one or more times.

3. The method of claim 1, wherein the at least one thioredoxin reductase inhibitor comprises auranofin.

4. The method of claim 1, wherein steps (1) and (2) are performed wholly or partially sequentially, and wherein the at least one composition is administered before or after the application of the alternating electric field has begun.

5. The method of claim 1, wherein the cancer cells are selected from the group consisting of hepatocellular carcinoma cells, glioblastoma cells, pleural mesothelioma cells, differentiated thyroid cancer cells, advanced renal cell carcinoma cells, ovarian cancers, breast cancers, pancreatic cancers, lung cancers, and combinations thereof.

6. The method of claim 1, further comprising the step of exposing the cancer cells to radiation therapy, wherein the radiation therapy is applied before step (1), after step (1), before step (2), and/or after step (2).

7. The method of claim 1, wherein the at least one composition further comprises at least one chemotherapeutic agent.

8. The method of claim 1, further comprising the step of administering a second composition to the cancer cells, wherein the first and second compositions are administered substantially simultaneously or wholly or partially sequentially.

9. The method of claim 8, wherein at least one of:
the first composition comprises at least one thioredoxin reductase inhibitor, and the second composition comprises at least one glutathione inhibitor;
the first composition comprises at least one glutathione inhibitor, and the second composition comprises at least one thioredoxin reductase inhibitor; and/or
the second composition comprises at least one chemotherapeutic agent.

10. A method of treating cancer in a subject, the method comprising the steps of:
(1) administering at least one composition to the subject, wherein the at least one composition comprises at least one thioredoxin reductase inhibitor and/or at least one glutathione inhibitor; and
(2) applying an alternating electric field to a target region of the subject.

11. The method of claim 10, wherein the at least one thioredoxin reductase inhibitor comprises auranofin.

12. The method of claim 10, wherein the at least one composition is orally or intravenously administered to the subject.

13. The method of claim 10, wherein at least one of:
the alternating electric field is applied at a frequency in a range of from about 50 kHz to about 1 MHz;
the alternating electric field has a field strength of at least about 1 V/cm in at least a portion of the cancer cells;
the period of time that the alternating electric field is applied is at least about 50% of at least about a 24 consecutive hour time period; and
steps (1) and (2) are repeated one or more times.

14. The method of claim 10, wherein steps (1) and (2) are performed substantially simultaneously.

15. The method of claim 10, wherein steps (1) and (2) are performed wholly or partially sequentially, and wherein the at least one composition is administered before or after the application of the alternating electric field has begun.

16. The method of claim 10, wherein the cancer cells are selected from the group consisting of hepatocellular carcinoma cells, glioblastoma cells, pleural mesothelioma cells, differentiated thyroid cancer cells, advanced renal cell carcinoma cells, ovarian cancers, breast cancers, pancreatic cancers, lung cancers, and combinations thereof.

17. The method of claim 10, further comprising the step of exposing at least the target region of the subject to radiation therapy, wherein the radiation therapy is applied before step (1), after step (1), before step (2), and/or after step (2).

18. The method of claim 10, further comprising the step of administering a second composition to the cancer cells, wherein the first and second compositions are administered substantially simultaneously or wholly or partially sequentially.

19. The method of claim 18, wherein at least one of:
the first composition comprises at least one thioredoxin reductase inhibitor, and the second composition comprises at least one glutathione inhibitor;
the first composition comprises at least one glutathione inhibitor, and the second composition comprises at least one thioredoxin reductase inhibitor; and/or
the second composition comprises at least one chemotherapeutic agent.

20. A method of reducing a volume of a tumor and/or preventing an increase in volume of the tumor, wherein the tumor is present in a body of a living subject and includes a plurality of cancer cells, the method comprising the steps of:
(1) administering at least one composition to the subject, wherein the at least one composition comprises at least one thioredoxin reductase inhibitor and/or at least one glutathione inhibitor; and
(2) applying an alternating electric field to a target region of the subject.
